(19) United States
(12) Patent Application Publication
(10) Pub. No.: US 2004/0138713 A1
(43) Pub. Date: Jul. 15, 2004

(54) External defibrillation and transcutaneous pacing device and methods for operating the device

(76) Inventors: Ronald E. Stickney, Edmonds, WA (US); Robert G. Walker, Bothell, WA (US); James W. Taylor, Sammamish, WA (US); David R. Hampton, Woodinville, WA (US); Tarek Z. Elabbady, Redmond, WA (US)

Correspondence Address: INGRASSIA FISHER & LORENZ, P.C., 7150 E. CAMELBACK, STE. 325, SCOTTSDALE, AZ, 85251, US

(21) Appl. No.: 10/713,177

(22) Filed: Nov. 13, 2003

Related U.S. Application Data
(60) Provisional application No. 60/426,122, filed on Nov. 13, 2002.

(51) Int. Cl. A61N 1/39

(52) U.S. Cl. 607/5

(57) ABSTRACT

An apparatus configured to provide a defibrillation shock or pacing stimuli to a patient and methods for controlling the apparatus are provided. The method includes obtaining and analyzing physical parameters of the patient to determine whether the patient has a heart condition appropriately treated with a defibrillation shock or pacing stimuli, if the appropriate treatment is pacing stimuli, automatically determining a magnitude of the pacing stimuli and supplying the pacing stimuli to the patient at the magnitude and at a pacing rate.

### CROSS-REFERENCES TO RELATED APPLICATIONS

This application claims the benefit of U.S. Provisional Application No. 60/426,122, filed on Nov. 13, 2002.

### FIELD OF THE INVENTION

The present invention generally relates to an external defibrillation and transcutaneous pacing device for supplying electrical therapy to a patient, and in particular, for delivering a defibrillation shock or pacing stimuli (pulses) to a patient.

### BACKGROUND OF THE INVENTION

A normal human heart pumping pattern is called normal sinus rhythm, and is regulated by the body's biological pacemaker within the upper right chamber of the heart, which is commonly referred to as the right atrium. This natural pacemaker, which is generally referred to as the sinoatrial (SA) node, sends electrical signals to the right and left ventricular muscles in the lower chambers of the heart. In certain circumstances, such as during a heart attack, the normal or sinus heartbeat rhythm may be adversely affected as a result of some type of malfunction in the heart's electrical control system. When this type of malfunction occurs, an abnormal heartbeat may result, causing the ventricular muscles to pump ineffectively, thus reducing the amount of blood pumped to the body. This abnormal heartbeat is generally referred to as an arrhythmia.

A particularly serious arrhythmia is known as ventricular fibrillation (VF), which is a malfunction characterized by rapid, uncoordinated cardiac movements replacing the normal contractions of the ventricular muscles. In this event, the ventricular muscles are not able to pump blood out of the heart. VF rarely terminates spontaneously, and is therefore a leading cause of sudden cardiac death. Thus, to aid in terminating VF, defibrillators are typically used to provide life-saving electrical shock therapy to persons experiencing VF giving the heart the chance to return to a normal rhythm.

Other serious arrhythmias have also been known to occur, such as an abnormally fast (tachycardia) or slow (bradycardia) heart rhythm. If tachycardia or bradycardia cause serious symptoms in the patient, pacing stimuli can be provided to the heart to terminate tachycardia or accelerate bradycardia. Tachycardia can also be terminated by a medium-strength shock in a procedure called synchronized cardioversion. If a patient in ventricular tachycardia (VT) is unconscious, a defibrillation shock may be applied to terminate the tachycardia.

At times, VF and other arrhythmias may occur in rapid succession of one another. For example, if the VF has been present for at least a few minutes prior to defibrillation, the first rhythm after defibrillation typically may be asystole (lack of cardiac activity), ventricular standstill (atrial activity without ventricular activity), or bradycardia. As another example, at times, if a patient in VF or VT is successfully defibrillated, it has been found that in some patients VF or VT may return due to the presence of the conditions that caused VF or VT initially. It has been found that early therapeutic intervention for serious heart arrhythmias is preferable in an attempt to prevent the loss of life. Thus, in the case of successive arrhythmias, both an external defibrillator and a transcutaneous pacing device, or a defibrillator having pacing capabilities, are preferable at the site to allow defibrillation and/or pacing therapy to begin with controlled timing relative to the defibrillation shock.

Currently, external defibrillators with transcutaneous pacing capability are commercially available. The external defibrillation function is commonly automated, providing assistance to users having various levels of medical training on to how to provide defibrillation shock to the patient. However, typically, the transcutaneous pacing function is not fully automated.

The current art also includes implanted pacemaker/cardioverter/defibrillator (PCD) devices. Implanted PCDs may pace in demand or non-demand mode. Generally, the physician to sets the pacemaker at a desired pacing current and pacing rate at the time of implantation. Some implanted pacemakers can adapt their pacing rate to the oxygen needs of the patient, for example increasing the pacing rate when the patient is exercising. However, implanted PCD devices are unsuitable for use in the context of emergency situations.

Therefore, there is a need for an external device that can automatically recognize whether a patient has a heart condition that is treatable with a defibrillation shock, pacing pulses or a non-electrotherapeutic treatment. Once it is recognized that pacing is appropriate, it is desirable that the device be able to automatically determine one or more of: a pacing current, a pacing rate, and a length of time for which to administer the pacing stimuli. In addition, it is desirable to implement automation in all types of external defibrillators, including fully automatic, semi-automatic, fully automated or semi-automated and manual defibrillators. Furthermore, other desirable features and characteristics of the present invention will become apparent from the subsequent detailed description of the invention and the appended claims, taken in conjunction with the accompanying drawings and this background of the invention.

### SUMMARY OF THE INVENTION

A method for controlling an electrotherapeutic device configured to provide a defibrillation shock or pacing stimuli to a patient is provided. The method includes the steps of obtaining and analyzing physical parameters of the patient to determine whether the patient has a heart condition for which an appropriate treatment is either a defibrillation shock or pacing stimuli; automatically determining a magnitude at which to supply the pacing stimuli, based, at least in part, upon the physical parameters, if the appropriate treatment is pacing stimuli; and supplying the pacing stimuli to the patient at the determined magnitude and at a pacing rate.

In another embodiment, an external medical device for supplying electroshock therapy to a patient is provided. The apparatus includes a plurality of electrodes, an energy storage device and a controller. The plurality of electrodes is configured to deliver a defibrillation shock or pacing stimuli to, and sense one or more physical parameters associated with, the patient. The energy storage device is coupled to the plurality of electrodes and configured to store a charge. The controller is coupled to the plurality of electrodes and the energy storage device, and configured to obtain and analyze physical parameters of the patient, automatically determine a magnitude at which to supply the pacing stimuli, based, at least in part, upon the physical parameters, and supply the pacing stimuli to the patient at the determined magnitude and at a pacing rate.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed description. The present invention will hereinafter be described in conjunction with the appended drawings, wherein like numerals denote like elements, and:

FIG. 1 is a simplified schematic view of an external defibrillator connected to a patient in accordance with an exemplary embodiment of the invention;

FIG. 2 is a simplified block diagram of an external defibrillator in accordance with an exemplary embodiment of the invention;

FIG. 3 is a flowchart for the method of operating the controller 40 of the external defibrillator of FIG. 2 in accordance with an exemplary embodiment of the invention;

FIG. 4 is a flowchart for the method of operating the controller 40 in FIG. 2 in accordance with an exemplary embodiment of the invention; and

FIG. 5 is a flowchart for the method of operating the controller 40 of another external defibrillator of FIG. 2 in accordance with an exemplary embodiment of the invention

### DETAILED DESCRIPTION OF A PREFERRED EMBODIMENT OF THE INVENTION

The following detailed description of the invention is merely exemplary in nature and is not intended to limit the scope, applicability, or configuration of the invention in any way. Rather, the following description provides a convenient illustration for implementing exemplary embodiments of the invention. Various changes to the described embodiments may be made in the function and arrangement of the elements described herein without departing from the scope of the invention.

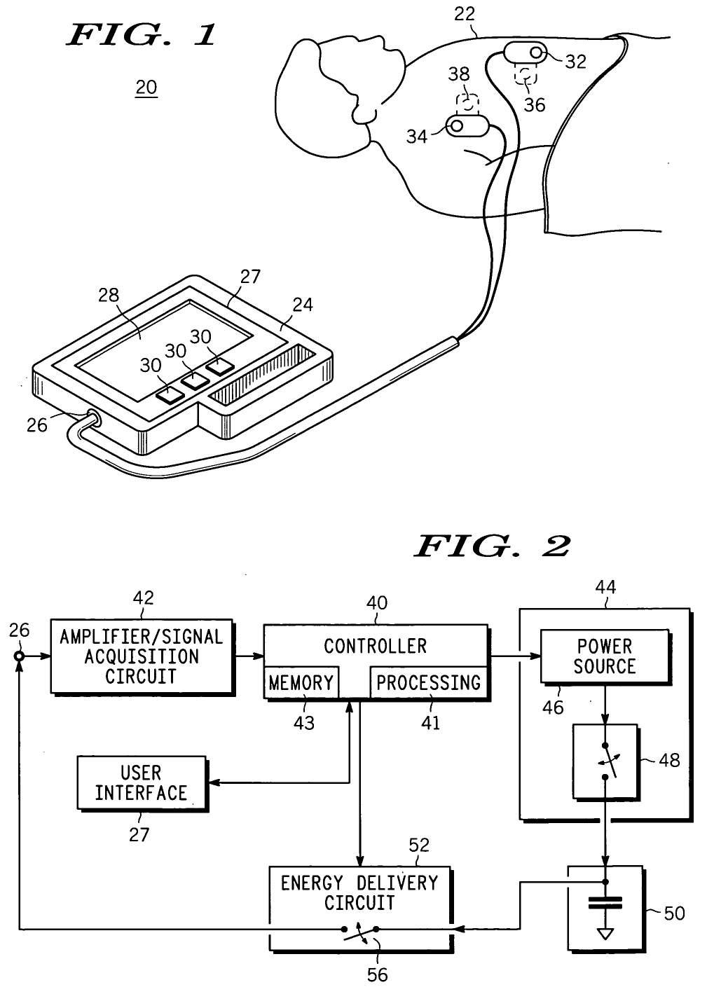

FIG. 1 is an external defibrillating system configured to be able to deliver both a defibrillation shock and a pacing stimulus to a patient. The system 20, includes, but is not limited to, an external defibrillator 24 having a connection port 26 that is configured to electrically connect defibrillator 24 to one or more electrodes 32,34. The external defibrillator 24 is preferably an automated external defibrillator (AED), that automatically diagnoses an electrocardiogram (ECG) from a patient 22, but can also be a fully automatic defibrillator that requires less human intervention than an AED. As used herein, an automatic or automated activity occurs without human intervention. Alternatively, the defibrillator 24 can be a manual monitor/defibrillator that may require more human intervention than an AED or fully automatic defibrillator. While many of the exemplary embodiments of the invention apply to all types of external defibrillators, some of the embodiments are only for specific types, such as embodiments only for automated defibrillators or only for manual monitor/defibrillators.

The external defibrillator 24 preferably includes a user interface 27 having a display 28 that is configured to visually present to the user various measured or calculated parameters associated with the patient 22 and/or other information to a user (not shown) of the external defibrillator 24. For example, the display 28 can be configured to visually present the transthoracic impedance, ECG and/or other physiological signals indicating the physical status of the patient 22, or instructions and/or commands, including prompts to perform cardiopulmonary resuscitation (CPR) therapy or other treatment instructions, to the user. The display 28 can also be configured to present visual alerts, flashing lights or warnings to the user. The user interface 27 can also include one or more input devices (e.g., switches or buttons) 30 that are configured to receive commands or information from the operator, such as, in the case of automated or manual defibrillators, a shock or pacing command. The user interface 27 can also include an audio system that provides an audio signal to the user in the form of voice prompts that deliver instructions or commands, monotonal, ascending, descending or quickening tones to indicate alerts or warnings, or any other suitable audio signals for communicating with the user. Additionally, the visual display 28 and audio signal may be configured to cooperate with one another.

The external defibrillator 24 is configured to determine and then generate a charge that is delivered to the patient 22 as the pacing stimulus or defibrillation shock with one or more electrodes 32, 34. The one or more electrodes 32, 34 may also be configured to sense one or more physiological and/or physical parameters of the patient 22 and supply signals representative of these parameters to the external defibrillator 24. The one or more physiological and/or physical parameters of the patient can include information about the patient's heart, blood, temperature and/or the like. More particularly, the sensed physical parameter can also be ECG data, heart rhythm data, heart rate data, the presence of electrical and/or mechanical capture of pacing stimuli, cardiac output data, blood flow data, a patient's level of perfusion, respiration data and/or any other physical parameter that is used in the art to assess the physical condition of a patient. As shown in phantom in FIG. 1, the external defibrillator 24 may additionally include one or more sensing electrodes 36,38 to sense the physiological and/or physical parameters. In either configuration, the signals provided by the one or more electrodes 32, 34 are preferably evaluated by the external defibrillator 24 to determine, among other things, whether a defibrillation shock, cardioversion shock or pacing stimuli should be applied to patient 22 in accordance with techniques known to those of ordinary skill in the art. The external defibrillator 24 can also evaluate the signals provided by the one or more electrodes 32,34 and/or one or more sensing electrodes 36,38 to determine the waveform parameters (e.g., voltage, current, energy and/or duration), magnitude and duration of the defibrillation shock, as well as the waveform parameters (e.g. current) and rate at which to provide pacing stimuli.

Referring to FIG. 2, a simplified block diagram of the circuitry that makes up the external defibrillator 24 is illustrated in accordance with an exemplary embodiment of the invention. The external defibrillator 24 in this embodiment preferably includes a controller 40 that is preferably in operable communication with user interface 27 (e.g., switches or buttons 30 and/or display 28 as shown in FIG. 1), an amplifier/signal acquisition circuit 42, and a charging mechanism 44 that can include a power source 46 and a switch 48, and may be operated under control of, the controller 40. It will be appreciated that the circuitry depicted in FIG. 2 is merely exemplary of a particular architecture, and that numerous other circuit architectures may be used to implement the operation of the external defibrillator 24. Each part of the circuitry will now be discussed.

The controller 40 may include, among other things, a processing unit 41. The processing unit 41 may be any one of numerous known general purpose processors or an application specific processor that operates in response to program instructions, which may be stored in any of various forms of memory storage, such as random access memory (RAM) or read-only memory (ROM), explained below. It will also be appreciated that the controller 40 may be implemented using various other circuits, not just a programmable processor. For example, digital logic circuits and analog signal processing circuits could also be used.

Controller 40 also includes a memory unit 43 that is in operable communication with processing unit 41 via a communications data bus (not shown). The memory unit 43 can include long-term memory (e.g., ROM), short-term memory (e.g., RAM) and medium-term memory (e.g., non-volatile RAM).

The long-term memory contains the operating system, software routines, and predetermined parameters. For instance, the data may include predetermined parameters that identify whether the sensed physical parameters indicate an arrhythmic heart condition, a shock or no-shock heart condition, or whether pacing is appropriate. The data may also include predetermined or preferred charge magnitude data, predetermined or preferred lengths of time for which to discharge energy storage device 50, and/or predetermined or preferred rate data at which to discharge energy storage device 50. Long-term memory can also include data that identify predetermined factors that indicate conditions that may benefit from certain further non-electrotherapeutic treatments. Such further treatments can include the administration of oxygen therapy, drug therapy, or CPR therapy, checking the patient for a pulse or for normal breathing, monitoring SaO₂, monitoring end tidal CO₂, or blood pressure levels, or any other non-electric treatment known in the art that is appropriately administered to a patient with an arrhythmic heart condition. Thus, long-term memory may also store both device-related data and physiological-related data. Long-term memory may be replaced when an upgrade is made available and installed.

Short-term memory (e.g., RAM) contains physiological signals for the current patient, measurements made on those signals, a log of events and therapy delivery that have occurred during the current case. Short-term memory can also receive and store the patient's sensed physical parameters and can store historical charge magnitude data, lengths of time and rate data the energy storage device 50 previously discharged to the patient.

Medium-term memory (e.g., nonvolatile RAM) stores a record of the current case for later review by clinicians. Data such as physiological signals, measurements, an event log that includes therapy delivered, and scene audio are typically stored in the medium-term memory as part of the case record. Medium-term memory typically is stored until the data are uploaded to a database of cases or until a new case is started.

It will be appreciated that above-mentioned scheme is merely exemplary of one scheme for storing operating software and software routines, and that various other storage schemes may be implemented. It will be appreciated that the memory unit 43 could be integrally formed as part of the controller 40 and/or processing unit 41, or could be part of a device or system that is physically separate from the external defibrillator 24.

Controller 40 makes treatment determinations based on the sensed physical parameters by comparing the sensed physical parameters or pacing parameter data to predetermined or historical data stored in the memory unit 43. For instance, the controller 40 is configured to determine whether a patient should receive a defibrillation shock, pacing stimuli or other treatment. The controller 40 is also configured to determine defibrillation parameters, such as an appropriate charge magnitude, rate of discharge and length of time for discharge of the energy storage device 50. The controller 40 can also determine pacing parameters, in the case of providing pacing stimuli, such as pacing current and pacing rate. The controller 40 is also configured to automatically determine updated pacing parameters based upon, for example, updated sensed physical parameters and/or whether the defibrillator 24 has previously delivered a defibrillation shock to the patient. Additionally, the controller 40 can be configured to continuously determine whether pacing should cease, in case pacing is no longer appropriate. The determination process is further detailed below.

If the controller 40 determines that a defibrillation shock is appropriate or that pacing stimuli should be delivered, controller 40 communicates with charging mechanism 44 to cause switch 48 to couple the power source 46 to one or more energy storage devices (e.g. capacitors) 50 to charge the one or more energy storage devices 50 to an appropriate charge magnitude. When the controller 40 decides to discharge the energy storage devices 50, the controller 40 communicates with an energy delivery circuit 52 and causes switch 56 to couple the one or more energy storage devices 50 to the connection port 26. The energy delivery circuit 52 can be implemented with any number of circuit configurations. For example, in a biphasic circuit, an H-bridge circuit can be used in accordance with the present invention. The discharge is delivered to the patient via electrodes 32, 34. In one embodiment, the defibrillation shock function and the pacing stimuli delivery function can use the same energy storage devices and/or energy delivery circuit. Alternatively, the defibrillation shock function and the pacing stimuli delivery function can each be coupled to its own energy storage device and/or associated energy delivery circuit.

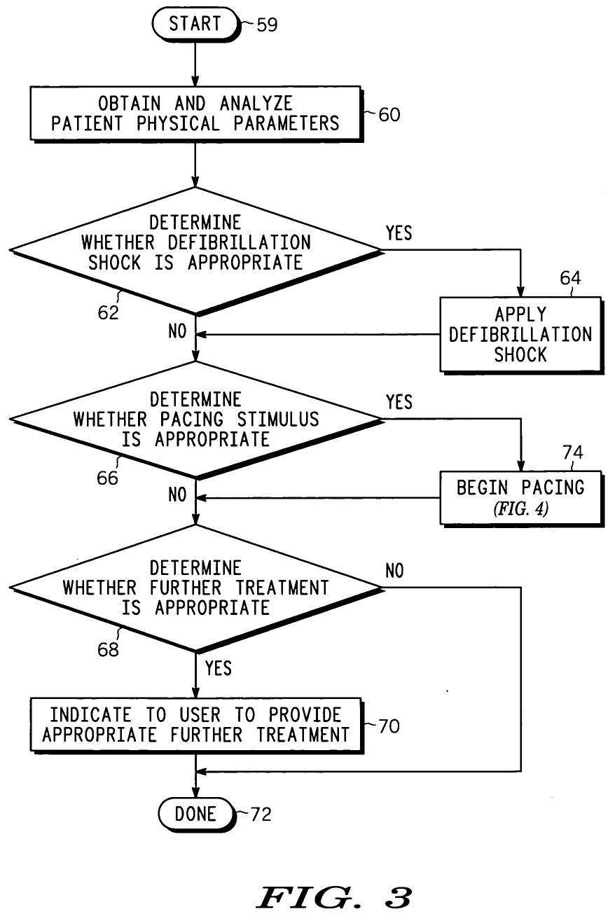

Having described a particular embodiment of the external defibrillating system 20, from a structural standpoint, and having generally described the overall functionality of the system 20, a more detailed description of a process implemented by the system 20 to automatically provide pacing stimuli to a patient will be provided. In doing so, reference should be made, as appropriate, to FIGS. 1 and 2, in combination with FIGS. 3-5, which illustrate several exemplary processes implemented by the system 20. It should be noted that the parenthetical reference numerals in the following description correspond to like reference numerals that are used to reference the flowchart blocks in FIGS. 3-5. Moreover, although the flowcharts depict “start” and “done” blocks, it will be appreciated that although a process may be complete or “done” it may be repeated as many times as desired.

Turning now to FIG. 3, a flowchart is provided illustrating the method of operating the controller 40 of the external defibrillator of FIG. 2 in accordance with an exemplary embodiment. In this embodiment, external defibrillator 24 is an automated external defibrillator. At anytime after the external defibrillator 24 is activated (59), by using any number of techniques such as activation of one of the input switches 30 shown in FIG. 1, one or more physical parameters of the patient are obtained and analyzed from the patient (60). The one or more physical parameters are sensed via the one or more electrodes 32,34,36,38. It will be appreciated that any number of physical parameters can be sensed. The signal or signals associated with the sensed physical parameters are preferably provided to the signal acquisition circuit 42 for preprocessing, digitization and/or amplification. Once the controller 40 receives the signals from the amplification/signal acquisition circuit 42 and analyzes the signals, the processing unit 41 determines whether or not the patient's physical parameters indicate a condition that should be treated with a defibrillation shock (62). This may be accomplished using any one of numerous techniques, but is preferably accomplished by making measurements on the sensed physical parameters and comparing those measurements to predetermined parameter data that indicate a shock or no-shock condition, which may be stored in memory unit 43. The controller 40 classifies the heart condition to determine whether or not defibrillation therapy should be delivered. Rhythms that are commonly treated with a defibrillation shock are ventricular fibrillation and pulseless ventricular tachycardia.

If the controller 40 classifies the heart condition as a shockable rhythm, then the external defibrillator 24 preferably determines an appropriate charge magnitude, causes the energy storage device 50 to charge to the charge magnitude, and then discharge a defibrillation shock to the patient via the one or more electrodes 32, 34 (64). The charge magnitude may be determined based, in part, on the sensed physical parameters or may be a preset charge magnitude. After the shock delivery, the controller 40 determines whether the patient should receive post-shock pacing stimuli (66). However, if the controller 40 classifies the heart condition as a non-shockable rhythm, then the controller 40 does not administer shock and instead determines whether pacing is appropriate, based in part on the sensed physical parameters (66).

Whether pacing is appropriate is determined by controller 40. Specifically, the processing unit 41 analyzes the sensed physical parameters, preferably by performing measurements on the sensed physical parameters, and compares measurements with predetermined parameters indicating abnormal heart conditions that may benefit from pacing, which may be stored in memory unit 43. For example, in one embodiment of the invention, one of the sensed physical parameters includes electrocardiogram (ECG) data, from which P waves, QRS complexes and various other data, including, among others, the patient's heart rate, can be extracted. The predetermined parameters include P waves at a normal rate, but a low ventricular heart rate (about 40 beats per minute (bpm) or less) with dissociated QRS complexes, which are parameters typically associated with third degree heart block (also called atrioventricular block). If the sensed physical parameters are compared to the predetermined parameters and are found to include P waves at a normal rate, but a low heart rate (about 40 bpm or less) and dissociated QRS complexes, then controller 40 determines that pacing is appropriate and potentially beneficial. Other predetermined parameters known in the art that indicate paceable heart conditions include, but are not limited to, severe bradycardia, ventricular asystole (or ventricular standstill) with P waves, second degree atrioventricular block, or low cardiac output, can be stored in the memory unit 43 as well. The predetermined parameters stored in memory unit 43 can also include tachycardia, which can be treated by overdrive pacing, in which the heart is paced for a short duration at a rate higher than the tachycardia rate in an attempt to terminate the tachycardia.

If a paceable heart condition is not indicated, then the controller 40 determines whether the patient would benefit from a non-electrotherapeutic treatment (68). For example, if the rhythm is asystole, or the patient does not have a pulse, or the patient does not have adequate cardiac output, the patient may benefit from CPR or drugs commonly used during resuscitation. Specifically, processing unit 41 analyzes the sensed physical parameters and compares them to predetermined treatment parameters, stored in memory unit 43, indicating non-electrotherapeutic treatment, thereby determining which non-electrotherapeutic treatment or treatments may be appropriate. If the controller 40 determines that non-electrotherapeutic treatment is appropriate, the controller 40 causes the user interface 27 to indicate to the care giver to provide further non-electrotherapeutic treatment (70), and the process is then complete (72). Should the controller 40 determine that non-electrotherapeutic treatment is inappropriate, the process is complete (72).

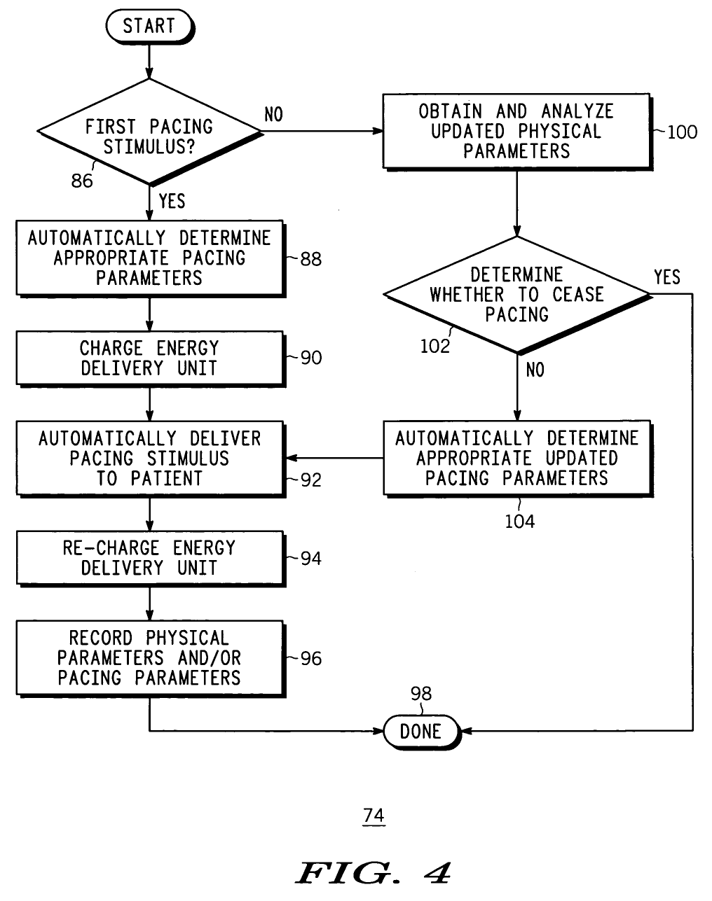

If a paceable heart condition is indicated, then the external defibrillator 24 automatically begins pacing (74). FIG. 4 is a flow chart that illustrates a method of operating controller 40 as described in the description of FIG. 3 in accordance with an embodiment of the invention. FIG. 4 further details Step 74 [Begin Pacing] in FIG. 3. As in FIG. 3, the external defibrillator 24 here is an automated external defibrillator. To begin pacing, the controller 40 determines whether or not the patient's heart has previously been provided pacing stimuli during the instant session (86). If the patient's heart has not previously been provided a pacing stimulus, the controller 40 automatically determines an initial pacing current and an initial pacing rate of, for example, about 80 per minute (88). The initial pacing current can be low (e.g., 40 milliAmperes [mA], with the intent of increasing current until electrical capture is detected), medium (e.g., 80 mA, with the intent of subsequently decreasing or increasing the current to find the lowest magnitude at which consistent electrical capture occurs), or high (e.g., 200 mA or more, with the intent of subsequently decreasing the current to find the lowest magnitude at which consistent electrical capture occurs). Alternatively, the initial desired pacing current and pacing rate can be preset or predetermined by the user.

The controller 40 then causes the energy storage device 50 to charge to the appropriate charge magnitude (90). The controller 40 then automatically causes the energy storage device 50 to discharge at the determined current magnitude at the determined pacing rate to thereby supply pacing stimuli to the patient (92). The energy storage device 50 is automatically recharged (94) and sensed physical parameters are then recorded (96). The sensed physical parameters are stored in memory unit 43. Alternatively, data determined in part from the sensed physical parameters, namely, the determined pacing parameters and/or ECG signals, transthoracic impedance or any other data providing cardiac output information, may be stored in the memory unit 43 after the discharge of the energy storage device 50. In yet another embodiment, the determined pacing parameters can be stored in the memory unit 43 at any time after the pacing parameter determinations are made. In still yet other embodiments, the sensed physical parameters or the data determined in part from the sensed physical parameters can be recorded at anytime after the physical parameters are sensed or anytime after the determinations based on the sensed physical parameters are made. The process is then complete (98).

Turning back to FIG. 4, if the patient's heart has previously been provided one or more pacing stimuli, controller 40 obtains updated one or more physical parameters from the patient (100). The updated one or more physical parameters are sensed via the one or more electrodes 32,34,36,38. The processing unit 41 then determines whether to cease pacing, based in part on the sensed physical parameters (102). The processing unit 41 compares the sensed physical parameters to data stored in the memory unit 43 that indicate unsuccessful electrical or mechanical capture, failure of improvement of cardiac output, detection of a shockable rhythm, or return of spontaneous circulation or any other indication that pacing should cease. Alternatively, the controller 40 can determine whether the patient has already received pacing stimuli for a threshold duration and if so, pacing should cease. In either case, if the controller 40 determines that pacing should cease, then the process is complete (98).

If the controller 40 or processing unit 41 determines that pacing should continue, the controller 40 automatically determines updated pacing parameters, based in part on the updated sensed physical parameters (104). Specifically, the processing unit 41 automatically compares the updated sensed physical parameter data to the stored sensed physical parameter data. The controller 40 automatically causes the energy storage device 50 to discharge according to the updated pacing parameters (92). Subsequently, the controller 40 automatically recharges the energy delivery unit (94) to prepare for the next pacing stimulus. The updated data are recorded in the controller 40 memory unit 41 (96) and the process is then complete (98).

It will be understood by those skilled in the art that one cycle of the steps in the process described in the flowchart of FIG. 4 illustrating an exemplary embodiment of the invention results in one pacing stimulus. Thus, the process as shown in FIG. 4 is repeated for every pacing stimulus that is delivered to the heart. The process in FIG. 4 may be used in conjunction with a non-demand or demand pacing function. In the case of non-demand pacing, if the desired pacing rate is 60 per minute, the process in FIG. 4 repeats once every second. However, in an embodiment that includes demand pacing, the defibrillator 24 may include additional sensing electrodes (not shown) and one or more timer circuits (not shown) that are in operable communication with the controller 40. The controller 40 continuously senses and monitors the patient's cardiac activity via the electrodes. The timer circuit and controller 40 operate to determine the time that elapses since the most recent delivered pacing stimulus, spontaneous QRS complex (preferably detected from the ECG signal), or cardiac pulse (preferably sensed from the impedance signal or other sensed physical parameter). If the timer reaches a threshold time period that is about the inverse of the pacing rate, the controller 40 causes the defibrillator 24 to perform the process in FIG. 4 and the timer is reset. Specifically, if a QRS complex is detected within a time interval that is equal to the inverse of the selected pacing rate, pacing stimulus is not delivered. Additionally, in demand pacing, pacing can cease if a timer measures a maximum threshold pacing duration time or if the timer measure a maximum threshold duration time for staying in demand pacing mode without delivery of a pacing stimulus.

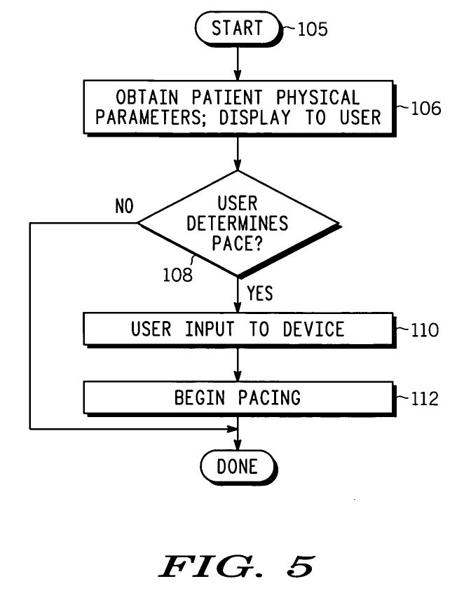

FIG. 5 provides a flowchart illustrating yet another embodiment in which the external defibrillator 24 is a manual monitor/defibrillator. At the beginning (105), the external defibrillator 24 obtains the patient's physical parameters via the electrodes 32, 34, 36, 38 (106), and displays the sensed physical parameters on user interface 27 (106). The user then determines, based in part on the sensed physical parameters, whether arrhythmia is indicated, and more specifically, whether pacing stimuli should be applied (108). If the user decides that pacing stimuli may be appropriate, the caregiver initiates pacing by providing an input to the external defibrillator 24, such as by manipulating a switch 30 (110). The external defibrillator 24 then automatically begins pacing (112) and initiates the steps described in the embodiment illustrated in FIG. 4.

In yet another embodiments, such as in the case of a manual monitor/defibrillator, an override option may be provided as an alternative to the fully automatic determination of the appropriate pacing current, predetermined rate and predetermined length of time, so that such determinations can be semi-automatic, where human intervention is employed to make one or more of the determinations, or manual, where all of the determinations are made by the caregiver. In another embodiment, the caregiver may select the initial pacing current and the monitor/defibrillator may automatically optimize the current, based in part, on sensed physical parameters.

Thus, an automatic pacing device is provided that automatically recognizes whether a patient has a heart condition that is treatable with a defibrillation shock, a pacing or a non-electrotherapeutic treatment. Once recognized, the device automatically determines a current, rate and length of time for which to administer the pacing stimuli. In addition, the invention may be implemented in all types of external defibrillators, including fully automatic, semi-automatic, fully automated or semi-automated and manual defibrillators.

While specific embodiments have been presented in the foregoing detailed description of the invention, it should be clear that a vast number of variations exist. It should also be appreciated that the exemplary embodiments are only examples, and are not intended to limit the scope, applicability, or configuration of the invention in any way. Rather, the foregoing detailed description will provide those skilled in the art with a convenient road-map for implementing an exemplary embodiment of the invention. It should be understood that various changes may be made in the function and arrangement of elements described in an exemplary embodiments without departing from the scope of the invention as set forth in the appended claims.

We claim:

1. A method for controlling an electrotherapeutic device configured to provide a defibrillation shock or pacing stimuli to a patient, comprising:
obtaining and analyzing physical parameters of the patient to determine whether the patient has a heart condition for which an appropriate treatment is either a defibrillation shock or pacing stimuli;
automatically determining a magnitude at which to supply the pacing stimuli, based, at least in part, upon the physical parameters, if the appropriate treatment is pacing stimuli; and
supplying the pacing stimuli to the patient at the determined magnitude and at a pacing rate.

2. The method of claim 1, wherein the step of obtaining and analyzing physical parameters of the patient comprises:
comparing the physical parameters to one or more predetermined parameters indicating severe bradycardia.

3. The method of claim 1, wherein the step of obtaining and analyzing physical parameters of the patient comprises:
comparing the physical parameters to one or more predetermined parameters indicating ventricular standstill.

4. The method of claim 1, wherein the step of obtaining and analyzing physical parameters of the patient comprises:
comparing the physical parameters to one or more predetermined parameters indicating second degree atrioventricular block.

5. The method of claim 1, wherein the step of obtaining and analyzing physical parameters of the patient comprises:
comparing the physical parameters to one or more predetermined parameter indicating third degree atrioventricular block.

6. The method of claim 1, wherein the step of obtaining and analyzing physical parameters of the patient comprises:
comparing the physical parameters to one or more predetermined parameter indicating low cardiac output.

7. The method of claim 1, wherein the step of obtaining and analyzing comprises:
determining whether a defibrillation shock has been delivered to the patient within a predetermined period of time.

8. The method of claim 1, further comprising:
obtaining and analyzing updated patient physical parameters.

9. The method of claim 8, further comprising:
automatically adjusting the magnitude and pacing rate to an updated magnitude and updated pacing rate based, in part, on the updated physical parameters; and
supplying the pacing stimuli to the patient at the updated magnitude and at the updated pacing rate.

10. The method of claim 8, further comprising:
identifying an indication to cease the pacing stimuli, based, in part, on the updated physical parameters; and
terminating the discharging of the energy device before the determined time, if an indication to cease the pacing stimuli is indicated.

11. The method of claim 10, wherein the step of identifying an indication to cease the pacing includes identifying no electrical capture.

12. The method of claim 10, wherein the step of identifying an indication to cease pacing includes identifying no mechanical capture.

13. The method of claim 10, wherein the step of identifying an indication to cease pacing includes identifying failure in improvement of cardiac output.

14. The method of claim 10, wherein the step of identifying an indication to cease pacing includes identifying adequate spontaneous circulation.

15. The method of claim 1 wherein the step of obtaining and analyzing physical parameters of the patient to determine whether the patient has a heart condition appropriately treated with a defibrillation shock or pacing stimuli includes determining whether the patient has a heart condition appropriately treated with a non-electrotherapeutic treatment.

16. The method of claim 15, further comprising:
indicating to a user that a non-electrotherapeutic treatment is needed.

17. The method of claim 16, wherein the step of indicating to a care provider that non-electrotherapeutic treatment is needed includes prompting the user to provide CPR therapy to the patient.

18. The method of claim 16, wherein the step of indicating to a care provider that non-electrotherapeutic treatment is needed includes prompting the user to provide drug therapy to the patient.

19. The method of claim 16, wherein the step of indicating to a care provider that non-electrotherapeutic treatment is needed includes prompting the user to provide oxygen therapy to the patient.

20. The method of claim 16, wherein the step of indicating to a care provider that non-electrotherapeutic treatment is needed includes prompting the user to monitor the patient's SaO₂ level.

21. The method of claim 16, wherein the step of indicating to a care provider that non-electrotherapeutic treatment is needed includes prompting the user to monitor the patient's blood pressure.

22. The method of claim 16, wherein the step of indicating to a care provider that non-electrotherapeutic treatment is needed includes prompting the user to monitor the patient's end tidal CO₂ level.

23. The method of claim 16, further comprising:
determining a physical status based, in part, on the patient's physical parameters; and
indicating the physical status to a user.

24. An external medical device for supplying electroshock therapy to a patient comprising:
a plurality of electrodes configured to deliver a defibrillation shock or pacing stimuli to, and sense one or more physical parameters associated with, the patient;
an energy storage device coupled to the plurality of electrodes and configured to store a charge; and
a controller coupled to the plurality of electrodes and the energy storage device, and configured to:
obtain and analyze physical parameters of the patient;
automatically determine a magnitude at which to supply the pacing stimuli, based, at least in part, upon the physical parameters; and
supply the pacing stimuli to the patient at the determined magnitude and at a pacing rate.

25. The device of claim 24, wherein the controller is further configured to:
obtain and analyze updated patient physical parameters;
automatically adjust the magnitude and pacing rate to an updated magnitude and updated pacing rate based, in part, on the updated physical parameters; and
supply pacing stimuli to the patient at the updated magnitude and at the updated rate.

26. The device of claim 25, wherein the controller is further configured to:
identify an indication to cease the pacing stimuli, based in part on the updated patient physical parameters; and
terminate the discharge of the energy device before the predetermined time, if an indication to cease the pacing stimuli is indicated.

27. The device of claim 24, wherein the controller is further configured to:
indicate to a care provider that further treatment is needed.

28. The device of claim 24, wherein the controller is further configured to:
determine a physical status based, in part, on the patient's physical parameters; and
indicate the physical status to a user.

29. The device of claim 24, further comprising:
a user interface in communication with the controller.

30. An external medical device for supplying electroshock therapy to a patient comprising:
a plurality of electrodes configured to deliver a defibrillation shock or pacing stimuli to, and sense one or more physical parameters associated with, the patient;
an energy storage device coupled to the plurality of electrodes and configured to store a charge; and
a controller coupled to the plurality of electrodes and the energy storage device, and configured to:
obtain and analyze physical parameters of the patient to determine whether the patient has a heart condition for which an appropriate treatment is either a defibrillation shock or pacing stimuli;
automatically determine a magnitude at which to supply the pacing stimuli, based, at least in part, upon the physical parameters; and
supply the pacing stimuli to the patient at the determined magnitude and at a pacing rate.

31. A method for controlling an electrotherapeutic device configured to provide a defibrillation shock or pacing stimuli to a patient, comprising:
obtaining and analyzing physical parameters of the patient to determine whether the patient has a heart condition for which an appropriate treatment is either a defibrillation shock or pacing stimuli;
automatically determining a magnitude at which to supply the pacing stimuli, based, at least in part, on whether the device previously provided a defibrillation shock to the patient, if the appropriate treatment is pacing stimuli; and
supplying the pacing stimuli to the patient at the determined magnitude and at a pacing rate.
